(19) United States
(12) Patent Application Publication
(10) Pub. No.: US 2011/0250252 A1
(43) Pub. Date: Oct. 13, 2011

(54) COLOURING TECHNIQUES

(76) Inventors: Leigh Canham, Worcestershire (GB); Armando Loni, Worcester (GB); Alastair Godfrey, Hampshire (GB)

(21) Appl. No.: 13/121,863

(22) PCT Filed: Sep. 30, 2009
(86) PCT No.: PCT/GB2009/051279

§ 371 Date: Jun. 2, 2011

(30) Foreign Application Priority Data
Sep. 30, 2008 (GB) 0817940.0

(51) Int. Cl. A61K 8/25; A23L 1/27; A61K 8/02; A61Q 1/02; A61Q 11/00

(52) U.S. Cl. 424/401; 424/63; 424/49; 426/262

(57) ABSTRACT

A method for masking the appearance of silicon in a range of compositions is described.

### FIELD OF THE INVENTION

This invention relates to the use of silicon in a range of compositions wherein the appearance of the silicon is masked.

### BACKGROUND OF THE INVENTION

There is a tendency to associate certain colours with certain flavours and foodstuffs and certain colours with specific consumer goods. Specific colours or specific mixtures of colours are frequently used by companies to help brand their consumer goods.

Also, in the consumer care and food industries, various methods are used to stabilise various ingredients and to control the timing and release of said ingredients. Such methods enable the protection of food components to ensure against nutritional loss and to mask or preserve flavours and aromas. Suitable methods of protection also increase the stability of vitamin or mineral supplements which are normally sensitive to light, UV radiation, metals, humidity, temperature and oxygen. Similar issues affect a range of consumer care products such as hair care compositions, oral hygiene compositions and cosmetics.

There is a continued need for alternative methods and/or products for protecting, controlling the release of, and/or masking the taste of ingredients in the consumer care and food industries without compromising the appearance of said products.

The present invention is based on the surprising finding that the colour of silicon may be modified and blended with other components of various compositions and not be distinguishable to the human eye. The present inventors have also found that the colour of the modified silicon may be retained over extended periods of time. In particular, this colour retention may be achieved in connection with the use of partially oxidised porous silicon, and especially partially oxidised mesoporous silicon.

### SUMMARY OF THE INVENTION

According to a first aspect of the present invention, there is provided a method for masking the appearance of particulate elemental silicon in a composition, comprising modifying the colour of the particulate elemental silicon so that the silicon is not distinguishable to the human eye when blended with the other components of the composition.

Preferably, the colour of the particulate elemental silicon is modified prior to blending with the other components.

The colour of the silicon particles may be modified by one or more of a range of techniques. For example, the silicon may be contacted with a suitable masking material in order to mask the appearance of the silicon and tailored to a suitable colour for the end product. The masking material may coat the silicon. When the silicon is selected from porous silicon, some, or all, or substantially all of the pores may be partially or completely filled with masking material, said masking material optionally being suitable for delivery to the human or animal body. In addition to the masking material, the silicon may be loaded with at least one further ingredient which is different from the masking material.

The at least one further ingredient and/or the masking material may be released after interaction with the human or animal body. For example, the release may be triggered by contact with gastrointestinal tract fluid such as saliva, gastric fluid, intestinal fluid or sweat. The release of the at least one further ingredient and/or the masking material may be triggered by one or more of a range of responses. These include, for example, contact with water of a particular temperature, for example, warm water during hair washing.

Loading the silicon with at least one further ingredient includes wherein the silicon is used to coat or partially coat one or more ingredients. For example, in food compositions, the silicon may be used to coat or partially coat breakfast cereals and the like or a product or products suitable for making beverages, such as coffee granules, coffee powder, tea, cocoa powder, chocolate powder. For example, when the ingredient is a microparticle (about 1-1000 μm in diameter), such as a powder or a small granule, or is a macroparticle (about 1 mm-20 mm), such as a granule or a typical cereal, said ingredient may be coated with silicon nanoparticles and/or microparticles.

The present inventors have also found that the use of partially oxidised porous silicon is particularly effective in retaining the colour of the masking material, at least in part, through the effects of photostabilisation or photoprotection. The present inventors have found that the presence of the partially oxidised surface results in the colour of the masking material (e.g. a white or colour pigment) not being unduly affected by the surface of the porous silicon which would otherwise comprise a hydride surface. The partially oxidised porous silicon serves to maintain the original hue of the masking material more effectively than other modified surfaces. As such, in a further aspect of the present invention, there is provided the use of partially oxidised porous silicon as a photostabiliser or photoprotector for a masking agent in a composition. There is also provided the use of partially oxidised porous silicon as a photostabiliser or photoprotector for a white or colour pigment. The modified porous silicon serves to assist in retaining the colour of the masking agent. Advantageously, the porous silicon may be mesoporous silicon.

The partially oxidised porous silicon may possess an oxide content corresponding to between about one monolayer of oxygen and a total oxide thickness of less than or equal to about 4.5 nm covering the entire skeleton.

### DETAILED DESCRIPTION OF THE INVENTION

### Silicon

As used herein, and unless otherwise stated, the term “silicon” refers to solid elemental silicon. For the avoidance of doubt, and unless otherwise stated, it does not include silicon-containing chemical compounds such as silica, silicates or silicones, although it may be used in combination with these materials.

The purity of the silicon may depend to some extent on the final application of the silicon. For example, the silicon may be about 95 to 99.99999% pure, for example about 96 to 99.9% pure. So-called metallurgical silicon which may be suitable in a range of applications, including foodstuffs, has a purity of about 98 to 99.5%.

The physical forms of silicon which are suitable for use in the method according to the present invention include one or more of amorphous silicon, single crystal silicon and polycrystalline silicon (including nanocrystalline silicon, the grain size of which is typically taken to be 1 to 100 nm) and including combinations thereof. Any of the above-mentioned types of silicon, which are suitable for use in the present invention, may be porosified to form porous silicon, which may be referred to as “pSi”. The silicon may be surface porosified or more substantially porosified. Suitable forms of porous silicon include mesoporous, microporous or macroporous silicon. Microporous silicon contains pores possessing a diameter less than 2 nm; mesoporous silicon contains pores having a diameter in the range of 2 to 50 nm; and macroporous silicon contains pores having a diameter greater than 50 nm.

The average pore diameter is measured using a known technique. Mesopore diameters are measured by very high resolution electron microscopy. This technique and other suitable techniques which include gas-adsorption-desorption analysis, small angle x-ray scattering, NMR spectroscopy or thermoporometry, are described by R. Herino in “Properties of Porous Silicon”, chapter 2.2, 1997. Micropore diameters are measured by xenon porosimetry, where the Xe¹²⁹ nmr signal depends on pore diameter in the sub 2 nm range. Macropore diameters are measured by electron microscopy. Alternative techniques include mercury porosimetry.

Other suitable forms of silicon include: submicron diameter polycrystalline particles; submicron diameter amorphous silicon particles; hollow silicon microparticles; agglomerated silicon nanoparticles; amorphous silicon coatings; micronised silicon alloys; micronised metallurgical grade silicon; cold pressed silicon particles.

The surface area and the size of the pores in the silicon may to some extent depend on what application the porous silicon is to be used for. For example, the BET surface area of the porous silicon is preferably in excess of 0.1 m²/g for microorganism entrapment, and preferably greater than 100 m²/g for biodegradability in intestinal fluid. The BET surface area is determined by a BET nitrogen adsorption method as described in Brunauer et al., J. Am. Chem. Soc., 60, p309, 1938. The BET measurement is performed using an Accelerated Surface Area and Porosimetry Analyser (ASAP 2400) available from Micromeritics Instrument Corporation, Norcross, Ga. 30093. The sample is outgassed under vacuum at 350° C. for a minimum of 2 hours before measurement.

The methods for making various forms of silicon which are suitable for use in the present invention are well known in the art.

In PCT/GB96/01863, the contents of which are incorporated herein by reference in their entirety, it is described how bulk crystalline silicon can be rendered porous by partial electrochemical dissolution in hydrofluoric acid based solutions. This etching process generates a silicon structure that retains the crystallinity and the crystallographic orientation of the original bulk material. Hence, the porous silicon formed is a form of crystalline silicon. Broadly, the method involves anodising, for example, a heavily boron doped CZ silicon wafer in an electrochemical cell which contains an electrolyte comprising a 20% solution of hydrofluoric acid in an alcohol such as ethanol, methanol or isopropylalcohol (IPA). Following the passing of an anodisation current with a density of about 50 mA cm ⁻², a porous silicon layer is produced which may be separated from the wafer by increasing the current density for a short period of time. The effect of this is to dissolve the silicon at the interface between the porous and bulk crystalline regions. Porous silicon may also be made using the so-called stain-etching technique which is another conventional method for making porous silicon. This method involves the immersion of a silicon sample in a hydrofluoric acid solution containing a strong oxidising agent. No electrical contact is made with the silicon, and no potential is applied. The hydrofluoric acid etches the surface of the silicon to create pores.

Mesoporous silicon may be generated from a variety of non-porous silicon powders by so-called “electroless electrochemical etching techniques”, as reviewed by K. Kolasinski in Current Opinions in Solid State & Materials Science 9, 73 (2005). These techniques include “stain-etching”, “galvanic etching”, “hydrothermal etching” and “chemical vapour etching” techniques. Stain etching results from a solution containing fluoride and an oxidant. In galvanic or metal-assisted etching, metal particles such as platinum are also involved. In hydrothermal etching, the temperature and pressure of the etching solution are raised in closed vessels. In chemical vapour etching, the vapour of such solutions, rather than the solution itself is in contact with the silicon. Mesoporous silicon can be made by techniques that do not involve etching with hydrofluoric acid. An example of such a technique is chemical reduction of various forms of porous silica as described by Z. Bao et al in Nature vol. 446 8th March 2007 p172-175 and by E. Richman et al. in Nano Letters vol. 8(9) p3075-3079 (2008). If this reduction process does not proceed to completion then the mesoporous silicon contains varying residual amounts of silica.

Following its formation, the porous silicon may be dried. For example, it may be supercritically dried as described by Canham in Nature, vol. 368, (1994), pp133-135. Alternatively, the porous silicon may be freeze dried or air dried using liquids of lower surface tension than water, such as ethanol or pentane, as described by Bellet and Canham in Adv. Mater, 10, pp487-490, 1998.

Silicon hydride surfaces may, for example, be generated by stain etch or anodisation methods using hydrofluoric acid based solutions. When the silicon, prepared, for example, by electrochemical etching in HF based solutions, comprises porous silicon, the surface of the porous silicon may or may not be suitably modified in order, for example, to improve the stability of the porous silicon in the composition. In particular, the surface of the porous silicon may be modified to render the silicon more stable in alkaline conditions. The surface of the porous silicon may include the external and/or internal surfaces formed by the pores of the porous silicon.

In certain circumstances, the stain etching technique may result in partial oxidation of the porous silicon surface. The surfaces of the porous silicon may therefore be modified to provide: silicon hydride surfaces; silicon oxide surfaces wherein the porous silicon may typically be described as being partially oxidised; or derivatised surfaces which may possess Si—O—C bonds and/or Si—C bonds. Silicon hydride surfaces may be produced by exposing the porous silicon to HF.

Silicon oxide surfaces may be produced by subjecting the silicon to chemical oxidation, photochemical oxidation or thermal oxidation, as described for example in Chapter 5.3 of Properties of Porous Silicon (edited by L. T. Canham, IEE 1997). PCT/GB02/03731, the entire contents of which are incorporated herein by reference, describes how porous silicon may be partially oxidised in such a manner that the sample of porous silicon retains some elemental silicon. For example, PCT/GB02/03731 describes how, following anodisation in 20% ethanoic HF, the anodised sample was partially oxidised by thermal treatment in air at 500° C. to yield a partially oxidised porous silicon sample.

Following partial oxidation, an amount of elemental silicon will remain. The silicon particles may possess an oxide content corresponding to between about one monolayer of oxygen and a total oxide thickness of less than or equal to about 4.5 nm covering the entire silicon skeleton. The porous silicon may have an oxygen to silicon atomic ratio between about 0.04 and 2.0, and preferably between 0.60 and 1.5. Oxidation may occur in the pores and/or on the external surface of the silicon.

Derivatised porous silicon is porous silicon possessing a covalently bound monolayer on at least part of its surface. The monolayer typically comprises one or more organic groups that are bonded by hydrosilylation to at least part of the surface of the porous silicon. Derivatised porous silicon is described in PCT/GB00/01450, the contents of which are incorporated herein by reference in their entirety. PCT/GB00/01450 describes derivatisation of the surface of silicon using methods such as hydrosilyation in the presence of a Lewis acid. In that case, the derivatisation is effected in order to block oxidation of the silicon atoms at the surface and so stabilise the silicon. Methods of preparing derivatised porous silicon are known to the skilled person and are described, for example, by J. H. Song and M. J. Sailor in Inorg. Chem. 1999, vol 21, No. 1-3, pp 69-84 (Chemical Modification of Crystalline Porous Silicon Surfaces). Derivitisation of the silicon may be desirable when it is required to increase the hydrophobicity of the silicon, thereby decreasing its wettability. Preferred derivatised surfaces are modified with one or more alkyne groups. Alkyne derivatised silicon may be derived from treatment with acetylene gas, for example, as described in “Studies of thermally carbonized porous silicon surfaces” by J. Salonen et al in Phys Stat. Solidi (a), 182, pp123-126, (2000) and “Stabilisation of porous silicon surface by low temperature photoassisted reaction with acetylene”, by S. T. Lakshmikumar et al in Curr. Appl. Phys. 3, pp185-189 (2003). Mesoporous silicon may be derivatised during its formation in HF-based electrolytes, using the techniques described by G. Mattei and V. Valentini in Journal American Chemical Society vol 125, p9608 (2003) and Valentini et al., Physica Status Solidi (c) 4 (6) p2044-2048 (2007).

Methods for making silicon powders such as silicon microparticles and silicon nanoparticles are well known in the art. Silicon microparticles are generally taken to mean particles of about 1 to 1000 μm in diameter and silicon nanoparticles are generally taken to mean particles possessing a diameter of about 100 nm and less. Silicon nanoparticles therefore typically possess a diameter in the range of about 1 nm to about 100 nm, for example about 5 nm to about 100 nm. Methods for making silicon powders are often referred to as “bottom-up” methods, which include, for example, chemical synthesis or gas phase synthesis. Alternatively, so-called “top-down” methods refer to such known methods as electrochemical etching or comminution (e.g. milling as described in Kerkar et al. J. Am. Ceram. Soc., vol. 73, pages 2879-2885, 1990). PCT/GB02/03493 and PCT/GB01/03633, the contents of which are incorporated herein by reference in their entirety, describe methods for making particles of silicon, said methods being suitable for making silicon for use in the present invention. Such methods include subjecting silicon to centrifuge methods, or grinding methods.

Other examples of methods suitable for making silicon nanoparticles include vaporation and condensation in a subatmospheric inert-gas environment. Various aerosol processing techniques have been reported to improve the production yield of nanoparticles. These include synthesis by the following techniques: combustion flame; plasma; laser ablation; chemical vapour condensation; spray pyrolysis; electrospray and plasma spray. Because the throughput for these techniques currently tends to be low, preferred nanoparticle synthesis techniques include: high energy ball milling; gas phase synthesis; plasma synthesis; chemical synthesis; sonochemical synthesis.

In the present invention, particle size distribution measurements, including the mean particle size (d₅₀/μm) of the silicon particles are measured using a Malvern Particle Size Analyzer, Model Mastersizer, from Malvern Instruments. A helium-neon gas laser beam is projected through a transparent cell which contains the silicon particles suspended in an aqueous solution. Light rays which strike the particles are scattered through angles which are inversely proportional to the particle size. The photodetector array measures the quantity of light at several predetermined angles. Electrical signals proportional to the measured light flux values are then processed by a microcomputer system, against a scatter pattern predicted from theoretical particles as defined by the refractive indices of the sample and aqueous dispersant to determine the particle size distribution of the silicon.

The preferred methods of producing silicon nanoparticles are described in more detail.

### High-Energy Ball Milling

High energy ball milling, which is a common top-down approach for nanoparticle synthesis, has been used for the generation of magnetic, catalytic, and structural nanoparticles, see Huang, “Deformation-induced amorphization in ball-milled silicon”, Phil. Mag. Lett., 1999, 79, pp305-314. The technique, which is a commercial technology, has traditionally been considered problematic because of contamination problems from ball-milling processes. However, the availability of tungsten carbide components and the use of inert atmosphere and/or high vacuum processes has reduced impurities to acceptable levels. Particle sizes in the range of about 0.1 to 1 μm are most commonly produced by ball-milling techniques, though it is known to produce particle sizes of about 0.01 μm.

Ball milling can be carried out in either “dry” conditions or in the presence of a liquid, i.e. “wet” conditions. For wet conditions, typical solvents include water or alcohol based solvents.

### Gas Phase Synthesis

Silane decomposition provides a very high throughput commercial process for producing polycrystalline silicon granules. Although the electronic grade feedstock (currently about $30/kg) is expensive, so called “fines” (microparticles and nanoparticles) are a suitable waste product for use in the present invention. Fine silicon powders are commercially available. For example, NanoSi™ Polysilicon is commercially available from Advanced Silicon Materials LLC and is a fine silicon powder prepared by decomposition of silane in a hydrogen atmosphere. The particle size is 5 to 500 nm and the BET surface area is about 25 m²/g. This type of silicon has a tendency to agglomerate, reportedly due to hydrogen bonding and Van der Waals forces.

### Plasma Synthesis

Plasma synthesis is described by Tanaka in “Production of ultrafine silicon powder by the arc plasma method”, J. Mat. Sci., 1987, 22, pp2192-2198. High temperature synthesis of a range of metal nanoparticles with good throughput may be achieved using this method. Silicon nanoparticles (typically 10-100 nm diameter) have been generated in argon-hydrogen or argon-nitrogen gaseous environments using this method.

### Chemical Synthesis

Solution growth of ultra-small (<10 nm) silicon nanoparticles is described in U.S. 20050000409, the contents of which are incorporated herein in their entirety. This technique involves the reduction of silicon tetrahalides such as silicon tetrachloride by reducing agents such as sodium napthalenide in an organic solvent. The reactions lead to a high yield at room temperature.

### Sonochemical Synthesis

In sonochemistry, an acoustic cavitation process can generate a transient localized hot zone with extremely high temperature gradient and pressure. Such sudden changes in temperature and pressure assist the destruction of the sonochemical precursor (e.g., organometallic solution) and the formation of nanoparticles. The technique is suitable for producing large volumes of material for industrial applications. Sonochemical methods for preparing silicon nanoparticles are described by Dhas in “Preparation of luminescent silicon nanoparticles: a novel sonochemical approach”, Chem. Mater., 10, 1998, pp 3278-3281.

### Mechanical Synthesis

Lam et al have fabricated silicon nanoparticles by ball milling graphite powder and silica powder, this process being described in J. Crystal Growth 220(4), p466-470 (2000), which is herein incorporated by reference in its entirety. Arujo-Andrade et al have fabricated silicon nanoparticles by mechanical milling of silica powder and aluminium powder, this process being described in Scripta Materialia 49(8), p773-778 (2003).

An alternative method for making porous silicon from nanoparticles includes exposing nanoparticulate elemental silicon to a pulsed high energy beam. The high energy beam may be a laser beam or an electron beam or an ion beam. Preferably, the high energy beam creates a condition wherein the elemental silicon is rapidly melted, foamed and condensed. Preferably, the high energy beam is a pulsed laser beam.

### Colour Modification

Various techniques may be used to modify the colour of the silicon particles in order that the silicon particles are not distinguishable to the human eye. Preferably, the colour of the composition is not significantly altered compared to when the silicon particles are not present. The colour of the silicon particles may be modified at the individual particle level by the use of a masking material which, in the case of porous silicon, fills at least some, or all, or substantially all of the pores and/or coats the outside of the silicon particles forming a capping layer. The masking material may fulfil the dual purpose of being at least one ingredient to be delivered to the human or animal body. The masking material may also comprise a layer of nanoparticles, effectively forming a coating or capping layer.

Suitable masking materials may include the use of high opacity materials, such as titania, which is white, or vividly coloured pigments or specific ingredients normally used in the composition. Examples of suitable masking materials include the following colouring agents which may be used in order to effect the desired colour: white—amydon; yellow—saffron, turmeric, annatto; green—parsley, spinach, sorrel; red—lycopene, carmine, sweet potato extract, red cabbage; blue—blueberry, mulberry extracts; purple—grape skin extract; caramel—heated corn syrup; brown—cinnamon; orange—beta-carotene. These colouring agents are particularly useful in food compositions. Other classes of colouring agents include natural biomolecules containing chromophores and non-linear optical properties. Examples include protein families such as carotenoids, rhodopsins, chlorophyll. A binding agent may be used in order to improve adhesion of the masking material to the particulate silicon. Suitable binding agents include natural binding agents, for example egg white or corn starch. Preferably the masking material is the same as an ingredient already present in the composition.

The appearance of the colour modified silicon in the composition is not distinguishable under conditions in which the particular composition would normally be viewed by the human eye during normal use. This includes normal daylight, bright direct sunlight and indoor lighting conditions.

The colour of the silicon particles may be modified at the individual particle level by controlling one or more of: the porosity, the surface roughness, the ordered arrangement of individual particles (and optionally the size of individual particles, which are typically nanoparticles), the silicon oxide content. Any of these modifications may also be carried out in combination with the inclusion of a masking material according to the present invention. In order to modify the colour of the particles by controlling the porosity, the porosity may advantageously be about 70 vol % to 95 vol %, for example about 70 to 80 vol %. Optionally, greater than 80 vol % of the pores may be filled with masking material. In order to modify the colour of the particles by controlling the arrangement of the individual particles in relation to each other, silicon nanoparticles may be formed into a self assembly of particles, such as a microparticle cluster, possessing a regular repeating pattern. Typically the number of nanoparticles in a cluster will be at least 100,000. In stand alone clusters forming a vividly coloured pure silicon microparticle powder the number of nanoparticles may be at least 10,000,000, for example 1,000,000,000 possessing a particle size of 1 to 100 nm, for example 2 to 10 nm. The nanoparticles may in film form, in which case the size ranges are of the same order as for the clusters though the number of nanoparticles is typically at least 1,000,000, for example at least 10,000,000.

Advantageously a certain amount of particles are colour modified. For example, at least 80 wt % of the silicon particles are colour modified. In order to determine the amount of particles which have been colour modified those particles are first separated from the rest of the formulation by filtration techniques. The filtered silicon particles are then washed and dried in a manner which does not change their colour, but removes any residual surface residues from the formulation. The colour uniformity of the silicon particles are then inspected in an imaging colorimeter of suitably high optical resolution. The colorimeter utilizes CCD sensor arrays to achieve independent capture of colour information at each spatial point within their field of view. If the colour of the silicon particles is modified by pore-filling or capping with suitable pigments, then the presence of those pigments can be quantified at the individual particle level by energy dispersive x-ray maps of chemical composition via inspection of the powder on a stage within a scanning electron microscope. The fractional area of the plan view image that lacks pigment provides an indication of the fraction of unmodified particles. If the outer surface of particles viewed at a higher magnification (exceeding ×50,000) still substantially retain their mesoporosity, then they have not been modified through pore-filling or capping with pigments.

### Ingredients

The silicon may be loaded with at least one ingredient which may be for delivery to a human or animal subject. The at least one ingredient may be the same as the masking material or may be different. The loading of the at least one ingredient may result in the capping of porous silicon.

Suitable ingredients include one or more of food ingredients (including those that are hydrophobic or degraded by the acidic conditions of the human or animal stomach), nutrients, hair care ingredients (including those that are light sensitive and/or whose efficacy benefits from improved retention on the scalp and/or hair follicles), cosmetic ingredients (including those that require segregation from other ingredients in the formulation), oral care ingredients (including those whose efficacy benefits from improved retention in the oral cavity).

The ingredient to be loaded with the porous silicon may be dissolved or suspended in a suitable solvent, and particles may be incubated in the resulting solution for a suitable period of time. Both aqueous and non-aqueous slips have been produced from ground silicon powder and the processing and properties of silicon suspensions have been studied and reported by Sacks in Ceram. Eng. Sci. Proc., 6, 1985, pp1109-1123 and Kerkar in J. Am. Chem. Soc. 73, 1990, pp2879-85. The removal of solvent will result in the ingredient penetrating into the pores of the porous silicon by capillary action, and, following solvent removal, the ingredient will be present in the pores. Preferred solvents, at least for use in connection with mesoporous silicon, are water, ethanol, and isopropyl alcohol, GRAS solvents and volatile liquids amenable to freeze drying.

Typically, the at least one ingredient is present in the range, in relation to the loaded silicon, of 0.01 to 90 wt %, for example 1 to 40 wt %, for example 20 to 50 wt % (optionally, in combination with about 70 % porosity) and for example 2 to 10 wt %.

Higher levels of loading, for example, at least about 15 wt % of the loaded ingredient based on the loaded weight of the porous silicon may be achieved by performing the impregnation at an elevated temperature. For example, loading may be carried out at a temperature which is at or above the melting point of the ingredient to be loaded. Quantification of gross loading may conveniently be achieved by a number of known analytical methods, including gravimetric, EDX (energy-dispersive analysis by x-rays), Fourier transform infra-red (FTIR), Raman spectroscopy, UV spectrophotometry, titrimetric analysis, HPLC or mass spectrometry. If required, quantification of the uniformity of loading may be achieved by techniques that are capable of spatial resolution such as cross-sectional EDX, Auger depth profiling, micro-Raman and micro-FTIR.

In connection with porous silicon, the loading levels can be determined by dividing the volume of the ingredient taken up during loading (equivalent to the mass of the ingredient taken up divided by its density) by the void volume of the porous silicon prior to loading multiplied by one hundred.

When present, the capping layer serves to encapsulate the silicon particles. Capping is advantageously carried out in connection with mesoporous silicon. When capped, the openings to the pores are sealed. Typically, the whole of the particle, or substantially all of the particle, is coated with the capping layer and the capping layer may be referred to herein as a bead. The capping layer at least seals the openings to the pores at the surface of the porous material, thus ensuring that the at least one loaded ingredient is retained. The capping layer, or bead, may encapsulate more than a single porous microparticulate material. The thickness of the capping layer may be about 0.1 to 50 μm in thickness, for example about 1 to 10 μm, for example about 1 to 5 μm. The capping layer may provide retention of an ingredient over a period of a few months to many months, for example up to about 5 years, followed by triggered release through site specific degradation which may occur in or on the human or animal body.

The thickness of the capping layer is measured by mechanically fracturing a number of the capped particles and examining their cross-sectional images in a high resolution scanning electron microscope, equipped with energy dispersive x-ray analysis (EDX analysis) of chemical composition. Alternatively, if the particle size distributions are measured accurately, before and after capping, then the average thickness of micron thick layer caps can be estimated. For relatively narrow particle size distributions and uniform coatings, if the density of the capping layer is known accurately, then accurate gravimetric measurements of weight increase that accompanies capping can also yield an average cap thickness.

There are various mechanisms by which the release of the loaded ingredient may be triggered. These include: biodegradation; mechanical means (e.g. forces exerted when brushing teeth); thermal means (e.g. change in temperature of water); optical irradiation (e.g. uv); microwave irradiation; chemical environment (e.g. change in pH).

Suitable methods for capping the silicon, particularly mesoporous silicon include: spray drying, fluidised bed coating, pan coating, modified microemulsion techniques, melt extrusion, spray chilling, complex coacervation, vapour deposition, solution precipitation, emulsification, supercritical fluid techniques, physical sputtering, laser ablation, very low temperature sintering and thermal evaporation.

### Compositions

The colour modified silicon is suitable for use in a range of compositions including consumer care compositions and food compositions. The consumer care compositions include hair care compositions, oral hygiene compositions and cosmetic compositions.

### (1) Food

The silicon may be used as a foodstuff in its own right and may, optionally, be loaded with one or more ingredients. The silicon may be loaded such that at least one ingredient is entrapped in the porous silicon and/or the silicon may coat or partially coat food particles and/or granules. These ingredients may be selected from one or more of: oxygen sensitive edible oils; minerals; oxygen sensitive fats including dairy fats; oil soluble ingredients; vitamins; fragrances or aromas; flavours; enzymes; probiotic bacteria; prebiotics; nutraceuticals; amino acids; herbal extracts; herbs; plant extracts; edible acids; salt; antioxidants; therapeutic agents. Typically, the one or more ingredients are present in the range, in relation to the loaded material, of 0.01 to 90 wt %, for example 1 to 40 wt % and for example 2 to 10 wt %.

The food may be in the form of a beverage or non-beverage. Suitable foods may comprise one or more of the following: meat; poultry; fish; vegetables; fruit; chocolate and confectionary; cereals and baked products including bread, cakes, biscuits, nutrition or cereal bars; pastry; pasta; dairy products such as milk, cream, butter, margarine, eggs, ice cream, cheese. The food may be in the form of any of the following: convenience meals; frozen food; chilled food; dried food; freeze dried food; rehydrated food; pickles; soups; dips; sauces.

Suitable beverages include alcoholic and non-alcoholic beverages. Particular examples of suitable drinks include water, for example bottled water; tea; coffee; cocoa; drinking chocolate; fruit juices and smoothies; wine; beer; ales; lager; spirits. The beverages may for example be in the form of powders or granules, including those which have been freeze dried, which are suitable for making instant coffee and tea and the like. These include coffee granules, coffee powder, coffee tablets, tea, cocoa powder, chocolate powder. Other suitable products include coffee oil and concentrates, for example, fruit juice concentrates.

The pH of the food is preferably such that the silicon does not dissolve in the food over a significant period of time and will thus afford an acceptable shelf-life. For example, for mesoporous silicon, the pH of the food is typically 2 to 6.

### Food Preparation

Methods for incorporating the silicon into food are numerous. Suitable mixing equipment is diverse and includes, for example, screw mixers, ribbon mixers and pan mixers. Other examples include high speed propeller or paddle mixers for liquid food or beverages; tumble mixers for dry powders; Z-blade mixers for doughs and pastes. Suitable grinding machines include hammer, disc, pin and ball millers. Extrusion is an important very high throughput (about 300-9000 kg/hr) technique for mixing and providing shape to foodstuffs and is suitable for use in the present invention. Cold and hot extruders may be used. These can be single or twin screw. Extruded foods include cereals, pasta, sausages, sugar or protein based products.

The total quantity of silicon present in the food, based on the total weight of the composition may be about 0.01 to 50 wt %, for example about 0.01 to 20 wt % and for example about 0.1 to 5 wt %.

### (2) Oral Hygiene Compositions

The silicon may be used in an oral hygiene composition such as a mouthwash or a dentifrice composition such as a toothpaste, tooth powder, lozenge, or oral gel. It may be present as an abrasive in addition to delivering one or more ingredients. The dentifrice composition will comprise constituents well known to one of ordinary skill; these may broadly be characterised as active and inactive agents. Active agents include anticaries agents such as fluoride, antibacterial agents, desensitising agents, antitartar agents (or anticalculus agents) and whitening agents. Inactive ingredients are generally taken to include water (to enable the formation of a water phase), detergents, surfactants or foaming agents, thickening or gelling agents, binding agents, efficacy enhancing agents, humectants to retain moisture, flavouring, sweetening and colouring agents, preservatives and, optionally further abrasives for cleaning and polishing. The oral gel may be of the type suitable for use in multi-stage whitening systems.

### Water Phase

The dentifrice composition typically comprises a water-phase which comprises an humectant. Water may be present in an amount of from about 1 to about 90 wt %, preferably from about 10 to about 60 wt %. Preferably, the water is deionised and free of organic impurities. Any of the known humectants for use in dentifrice compositions may be used. Suitable examples include sorbitol, glycerin, xylitol, propylene glycol. The humectant is typically present in an amount of about 5 to 85 wt % of the dentifrice composition.

### Anticaries Agent

The dentifrice composition according to the present invention may comprise an anticaries agent, such as a source of fluoride ions. The source of fluoride ions should be sufficient to supply about 25 ppm to 5000 ppm of fluoride ions, for example about 525 to 1450 ppm. Suitable examples of anticaries agents include one or more inorganic salts such as soluble alkali metal salts including sodium fluoride, potassium fluoride, ammonium fluorosilicate, sodium fluorosilicate, sodium monofluorophosphate, and tin fluorides such as stannous fluoride.

### Antitartar Agents

Any of the known antitartar agents may be used in the dentifrice compositions according to the present invention. Suitable examples of antitartar agents include pyrophosphate salts, such as dialkali or tetraalkali metal pyrophosphate salts, long chain polyphosphates such as sodium hexametaphosphate and cyclic phosphates such as sodium trimetaphosphate. These antitartar agents are included in the dentifrice composition at a concentration of about 1 to about 5 wt %.

### Antibacterial Agents

Any of the known antibacterial agents may be used in the compositions of the present invention. For example, these include non-cationic antibacterial agents such as halogenated diphenyl ethers, a preferred example being triclosan (2,4,4′-trichloro-2′-hydroxydiphenyl ether). The antibacterial agent(s) may be present in an amount of about 0.1 to 1.0 wt % of the dentifrice composition, for example about 0.3 wt %.

### Other Abrasive Agents

The silicon can be used as the sole abrasive in preparing the dentifrice composition according to the present invention or in combination with other known dentifrice abrasives or polishing agents. Commercially available abrasives may be used in combination with the silicon, e.g. porous silicon, and include silica, aluminium silicate, calcined alumina, sodium metaphosphate, potassium metaphosphate, calcium carbonate, calcium phosphates such as tricalcium phosphate and dehydrated dicalcium phosphate, aluminium silicate, bentonite or other siliceous materials, or combinations thereof.

### Flavours

The dentifrice composition of the present invention may also contain a flavouring agent. Suitable examples include essential oils such as spearmint, peppermint, wintergreen, sassafras, clove, sage, eucalyptus, majoram, cinnamon, lemon, lime, grapefruit, and orange. Other examples include flavouring aldehydes, esters and alcohols. Further examples include menthol, carvone, and anethole.

### Thickening Agents

The thickening agent may be present in the dentifrice composition in amounts of about 0.1 to about 10% by weight, preferably about 0.5 to about 4% by weight. Thickeners used in the compositions of the present invention include natural and synthetic gums and colloids, examples of which include xanthan gum, carrageenan, sodium carboxymethyl cellulose, starch, polyvinylpyrrolidone, hydroxyethylpropyl cellulose, hydroxybutyl methyl cellulose, hydroxypropylmethyl cellulose, and hydroxyethyl cellulose. Suitable thickeners also include inorganic thickeners such as amorphous silica compounds including colloidal silica compounds.

### Surfactants

Surfactants may be used to achieve increased prophylactic action and render the dentifrice compositions more cosmetically acceptable. The surfactant is typically present in the dentifrice compositions according to the present invention in an amount of about 0.1 to about 5 wt %, preferably about 0.5 to about 2 wt %. The dentifrice compositions according to the present invention may comprise one or more surfactants, which may be selected from anionic, non-ionic, amphoteric and zwitterionic surfactants. The surfactant is preferably a detersive material, which imparts to the composition detersive and foaming properties. Suitable examples of surfactants are well known to an ordinary skilled person and include water-soluble salts of higher fatty acid monoglyceride monosulfates, such as the sodium salt of the monosulfated monoglyceride of hydgrogenated coconut oil fatty acids, higher alkyl sulfates such as sodium lauryl sulfate, alkyl aryl sulfonates such as sodium dodecyl benzene sulfonate, higher alkyl sulfoacetates, sodium lauryl sulfoacetate, higher fatty acid esters of 1,2-dihydroxy propane sulfonate, and the substantially saturated higher aliphatic acyl amides of lower aliphatic amino carboxylic acid compounds, such as those having 12 to 16 carbons in the fatty acid, alkyl or acyl radicals. Further examples include N-lauroyl sarcosine, and the sodium, potassium, and ethanolamine salts of N-lauroyl, N-myristoyl, or N-palmitoyl sarcosine.

### Efficacy Enhancing Agents

One or more efficacy enhancing agents for any antibacterial, antitartar or other active agent within the dentifrice composition may also be included in the dentifrice composition. Suitable examples of efficacy enhancing agents include synthetic anionic polycarboxylates. Such anionic polycarboxylates may be employed in the form of their free acids or partially, or more preferably, fully neutralized water soluble alkali metal (e.g. potassium and preferably sodium) or ammonium salts. Preferred are 1:4 to 4:1 copolymers of maleic anhydride or acid with another polymerizable ethylenically unsaturated monomer, preferably methylvinylether/maleic anhydride having a molecular weight (M.W.) of about 30,000 to about 1,800,000.

When present, the efficacy enhancing agent, for example the anionic polycarboxylate, is used in amounts effective to achieve the desired enhancement of the efficacy of any antibacterial, antitartar or other active agent within the dentifrice composition. Generally, the anionic polycarboxylate(s) are present within the dentifrice composition from about 0.05 wt % to about 4 wt %, preferably from about 0.5 wt % to about 2.5 wt %.

### Other Ingredients

Various other materials may be incorporated in the dentifrice compositions of this invention, including: preservatives; silicones; desensitizers, such as potassium nitrate; whitening agents, such as hydrogen peroxide, calcium peroxide and urea peroxide; and chlorophyll compounds. Some toothpastes include bicarbonate in order to reduce the acidity of dental plaque. These additives, when present, are incorporated in the dentifrice composition in amounts which do not substantially adversely affect the desired properties and characteristics.

### Preparation of the Dentifrice Composition

Suitable methods for making the dentifrice compositions in accordance with the present invention include the use of high shear mixing systems under vacuum. In general, the preparation of dentifrices is well known in the art. U.S. Pat. No. 3,980,767, U.S. Pat. No. 3,996,863, U.S. Pat. No. 4,358,437, and U.S. Pat. No. 4,328,205, the contents of which are hereby incorporated by reference in their entirety, describe suitable methods for making dentifrice compositions.

For example, in order to prepare a typical dentifrice composition according to the present invention, an humectant may be dispersed in water in a conventional mixer under agitation. Organic thickeners are combined with the dispersion of humectant along with: any efficacy enhancing agents; any salts, including anticaries agents such as sodium fluoride; and any sweeteners. The resultant mixture is agitated until a homogeneous gel phase is formed. One or more pigments such as titanium dioxide may be combined with the gel phase along with any acid or base required to adjust the pH. These ingredients are mixed until an homogenous phase is obtained. The mixture is then transferred to a high speed/vacuum mixer, wherein further thickener and surfactant ingredients may be combined with the mixture. Any abrasives may be combined with the mixture to be used in the composition. Any water insoluble antibacterial agents, such as triclosan, may be solubilized in the flavour oils to be included in the dentifrice composition and the resulting solution is combined along with the surfactants with the mixture, which is then mixed at high speed for about 5 to 30 minutes, under vacuum of from about 20 to 50 mm of Hg. The resultant product is typically an homogeneous, semi-solid, extrudable paste or gel product.

The pH of the dentifrice composition is typically such that the silicon will not dissolve in the composition over a significant period of time and will thus afford an acceptable shelf-life. The pH of the dentifrice composition is typically less than or equal to about 9 and preferably, particularly for compositions other than powders such as toothpastes, less than or equal to about 7. The lower limit of pH may typically be about 3.5 or about 4. In particular, the pH may be about 3.5 or about 4 when the dentifrice composition is a gel, such as those used in multi-stage whitening systems.

The abrasivity of the dentifrice compositions made according to the present invention, can be determined by means of Radioactive Dentine Abrasion (RDA) values as determined according to the method recommended by the American Dental Association, as described by Hefferren, J. Dental Research, vol. 55 (4), pp 563-573, (1976) and described in U.S. Pat. No. 4,340,583, U.S. Pat. No. 4,420,312 and U.S. Pat. No. 4,421,527, the contents of which are contained herein by reference in their entirety. In this procedure, extracted human teeth are irradiated with a neutron flux and subjected to a standard brushing regime. The radioactive phosphorus 32 removed from the dentin in the roots is used as the index of the abrasion of the dentifrice tested. A reference slurry containing 10 g of calcium pyrophosphate in 15 ml of a 0.5% aqueous solution of sodium carboxymethyl cellulose is also measured and the RDA of this mixture is arbitrarily taken as 100. The dentifrice composition to be tested is prepared as a suspension at the same concentration as the pyrophosphate and submitted to the same brushing regime. The RDA of the dentifrice compositions according to the present invention may lie in the range of about 10 to 150, for example less than about 100, for example, less than about 70.

The pellicle cleaning ratio (PCR) of the dentifrice compositions of the present invention is a measurement of the cleaning characteristics of dentifrices and generally may range from about 20 to 150 and is preferably greater than about 50.

The PCR cleaning values can be determined by a test described by Stookey et al., J. Dental Research, vol. 61 (11), pp 1236-9, (1982). Cleaning is assessed in vitro by staining 10 mm² bovine enamel specimens embedded in resin, which are acid etched to expedite stain accumulation and adherence. The staining is achieved with a broth prepared from tea, coffee and finely ground gastric mucin dissolved into a sterilized trypticase soy broth containing a 24-hour Sarcina lutea turtox culture. After staining, the specimens are mounted on a V-8 cross-brushing machine equipped with soft nylon toothbrushes adjusted to 150 g tension on the enamel surface. The specimens are then brushed with the dentifrice composition to be tested and a calcium pyrophosphate standard which comprises 10 g of calcium pyrophosphate in 50 ml of 0.5% aqueous solution of sodium carboxymethyl cellulose. The specimens are brushed with dentifrice slurries consisting of 25 g of toothpaste in 40 g of deionized water, for 400 strokes. The specimens are cleaned with Pennwalt pumice flour until the stain is removed. Reflectance measurements are taken using a Minolta Colorimeter using the standard Commission Internationale de I'Eclairage (CIE) L* a* b* scale in order to measure the colour of the specimens before and after brushing.

The cleaning efficiency of the dentifrice compositions according to the present invention, which is a measure of the ratio of PCR/RDA, may lie in the range from about 0.5 to about 2.0, and may be greater than about 1.0 for example greater than about 1.5.

### (3) Hair Care Compositions

The term hair care composition as used herein includes shampoos, gels, creams, conditioners (including leave-on conditioners), combined shampoo/conditioners, hair dyes, mousses, foams, waxes, crème rinses, masks, muds, semi-solid structured styling pastes (also known as putties), styling sprays, hot oil treatments, rinses, lotions, all suitable for use on the hair of humans and animals, particularly on human hair, especially hair on the human head. The general constituents of these compositions are well known to the skilled person.

The pH of the hair care composition is advantageously such that the silicon does not dissolve in the composition over a significant period of time and will thus afford an acceptable shelf-life. The pH of the hair care composition is typically less than about 7.5 (though may be as high as about 8.5) and preferably less than or equal to about 7, for example less than or equal to about 6 and may be less than about 4.6. Most commercially available shampoos are, for example, about pH 5-6.5 and the pH of the hair care composition may lie in this range. The lower limit of pH may be about 2. For mesoporous silicon, a suitable pH range may be 2 to 6. For use in higher pH environments, such as up to about pH 8.5 the porous silicon may advantageously be stabilised, for example, by partial oxidation. This may be achieved by heating the porous silicon to about 500° C. over 1 hour in air or an oxygen-rich atmosphere.

Shampoos typically comprise water, surfactant, plus a host of optional further constituents. Water may be present in an amount of about 25% to about 99 wt %, for example about 50% to about 98 wt % based on the weight of the total composition.

Shampoo formulations typically contain high concentrations of surfactants, e.g. up to about 50 wt % based on the total weight of the shampoo. Surfactants may provide a number of functions. For example, they make the removal of dirt easier by reducing the surface tension between the water and the greasy matter on the hair. Any foam produced by the surfactant may hold the dirt in it, and prevent it from being re-deposited on the hair. Surfactants may stabilise the shampoo mixture, and help retain the other ingredients in solution. They may also thicken the shampoo and make it easier to use. Shampoos may contain several surfactants which may provide different types of cleaning, according to the type of hair. One commonly used surfactant is ammonium lauryl sulphate, another is ammonium laureth sulphate, which is milder. Many of the ingredients in shampoos are traditionally soft organic materials.

Most modern shampoos may contain conditioning agent. Other typical ingredients include lather boosters, viscosity modifiers and additives for controlling the pH. The pH of commercially available shampoos may vary quite widely, for example, some shampoos are formulated to be acidic, e.g. about pH 3.5-4.5. Other ingredients may include preservative such as sodium benzoate or parabens. Aesthetic ingredients include colours, perfumes, pearlescing agents.

The hair care compositions according to the present invention may comprise one or more surfactants. The surfactant may be selected from any of a wide variety of anionic, amphoteric, zwitterionic and non-ionic surfactants. The surfactant may be detersive. The amount of surfactant in, for example, the shampoo composition may be from 1 to 50 wt %, for example from 3 to 30 wt %, for example from 5% to 20 wt % based on the total weight of the composition.

Suitable anionic surfactants include alkyl sulphates, alkyl ether sulphates, alkaryl sulphonates, alkanoyl isethionates, alkyl succinates, alkyl sulphosuccinates, N-alkoyl sarcosinates, alkyl phosphates, alkyl ether phosphates, alkyl ether carboxylates, alpha-olefin sulphonates and acyl methyl taurates, for example, their sodium, magnesium ammonium and mono-, di- and triethanolamine salts. The alkyl and acyl groups may contain from 8 to 18 carbon atoms and may be unsaturated. The alkyl ether sulphates, alkyl ether phosphates and alkyl ether carboxylates may contain from 1 to 10 ethylene oxide or propylene oxide units per molecule, and may contain 2 to 3 ethylene oxide units per molecule.

Particular examples of suitable anionic surfactants include sodium lauryl sulphate, sodium lauryl ether sulphate, ammonium lauryl sulphosuccinate, ammonium lauryl sulphate, ammonium lauryl ether sulphate, sodium dodecylbenzene sulphonate, triethanolamine dodecylbenzene sulphonate, sodium cocoyl isethionate, sodium lauroyl isethionate, sodium N-lauryl sarcosinate, and mixtures thereof.

Examples of anionic detersive surfactants which may provide cleaning and lather performance to the composition include sulfates, sulfonates, sarcosinates and sarcosine derivatives.

The hair care composition may also include co-surfactants, to help impart aesthetic, physical or cleansing properties to the composition. Suitable examples include amphoteric, zwitterionic and/or non-ionic surfactants, which can be included in an amount ranging up to about 10 wt % based on the total weight of the shampoo composition. Examples of amphoteric or zwitterionic surfactants include alkyl amine oxides, alkyl betaines, alkyl amidopropyl betaines, alkyl sulphobetaines (sultaines), alkyl glycinates, alkyl carboxyglycinates, alkyl amphopropionates, alkylamphoglycinates, alkyl amidopropyl hydroxysultaines, acyl taurates and acyl glutamates, wherein the alkyl and acyl groups have from 8 to 19 carbon atoms. Typical amphoteric and zwitterionic surfactants for use in shampoos of the invention include lauryl amine oxide, cocodimethyl sulphopropyl betaine and lauryl betaine, cocamidopropyl betaine, sodium cocamphopropionate, and mixtures thereof.

Suitable non-ionic surfactants include condensation products of aliphatic (C₈ to C₁₈) primary or secondary linear or branched chain alcohols or phenols with alkylene oxides, usually ethylene oxide and generally having from 6 to 30 ethylene oxide groups. Other suitable non-ionic surfactants include mono- or di-alkyl alkanolamides. Examples include coco mono- or di-ethanolamide and coco mono-isopropanolamide.

Further non-ionic surfactants which can be included in shampoo compositions of the invention are the alkyl polyglycosides (APGs).

Further surfactant may also be present as emulsifier for emulsified components of the composition, e.g. emulsified particles of silicone. This may be the same surfactant as the anionic surfactant or the co-surfactant, or may be different. Suitable emulsifying surfactants are well known in the art and include anionic and non-ionic surfactants. Examples of anionic surfactants used as emulsifiers for materials such as silicone particles are alkylarylsulphonates, e.g., sodium dodecylbenzene sulphonate, alkyl sulphates e.g., sodium lauryl sulphate, alkyl ether sulphates, e.g., sodium lauryl ether sulphate nEO, where n is from 1 to 20 alkylphenol ether sulphates, e.g., octylphenol ether sulphate nEO where n is from 1 to 20, and sulphosuccinates, e.g., sodium dioctylsulphosuccinate.

Examples of non-ionic surfactants used as emulsifiers for materials such as silicone particles are alkylphenol ethoxylates, e.g., nonylphenol ethoxylate nEO, where n is from 1 to 50, alcohol ethoxylates, ester ethoxylates, e.g., polyoxyethylene monostearate where the number of oxyethylene units is from 1 to 30.

The hair care composition may also include one or more conditioning agents. As used herein, the term “conditioning agent” includes any material which is used to give a particular conditioning benefit to hair and/or the scalp or skin. For example, in shampoo compositions for use in washing hair, suitable materials are those which deliver one or more benefits relating to shine, softness, combability, wet-handling, anti-static properties, protection against damage, body, volume, stylability and manageability.

Conditioning agents for use in the present invention include emulsified silicones, used to impart, for example, wet and dry conditioning benefits to hair such as softness, smooth feel and ease of combability. The conditioning agent may be present in a level of from about 0.01 wt % to about 25 wt %, for example about 0.05 to about 10 wt %, for example about 0.1 to 5 wt % based on the total weight of the composition. The lower limit may be determined by the minimum level to achieve conditioning and the upper limit by the maximum level to avoid making the hair and/or skin unacceptably greasy. About 1 wt % is typically suitable.

A further class of silicones for inclusion in shampoos and conditioners of the invention are amino functional silicones. By “amino functional silicone” is meant a silicone containing at least one primary, secondary or tertiary amine group, or a quaternary ammonium group.

A further class of conditioning agents are peralkyl and peralkenyl hydrocarbon materials, used to enhance the body, volume and stylability of hair. Suitable materials include polyisobutylene materials available from Presperse, Inc. The amount of per-alkyl or peralkenyl hydrocarbon material incorporated into the compositions of the invention may depend on the level of body and volume enhancement desired and the specific material used. A suitable amount is from 0.01 to about 10 wt % by weight of the total composition. The lower limit is determined by the minimum level to achieve the body and volume enhancing effect and the upper limit by the maximum level to avoid making the hair unacceptably stiff. An amount of per-alk(en)yl hydrocarbon material of from 0.5 to 2 wt % of the total composition is a suitable level.

A cationic deposition polymer is an ingredient which may be included in shampoo compositions of the invention, for enhancing conditioning performance of the shampoo. By “deposition polymer” is meant an agent which enhances deposition of active ingredients and/or conditioning components (such as silicones) from the shampoo composition onto the intended site during use, i.e. the hair and/or the scalp.

The deposition polymer may be a homopolymer or be formed from two or more types of monomers. The molecular weight of the polymer may typically be at least 10,000, for example, in the range 100,000 to about 2,000,000. The polymers will have cationic nitrogen containing groups such as quaternary ammonium or protonated amino groups, or a mixture thereof. The cationic amines can be primary, secondary or tertiary amines.

As further optional components for inclusion in the hair care compositions of the invention one or more of the following may be included: pH adjusting agents, viscosity modifiers, pearlescers, opacifiers, suspending agents, preservatives, colouring agents, dyes, proteins, herb and plant extracts, and other moisturising and/or conditioning agents.

Any viscosity modifier suitable for use in hair care compositions may be used herein. Generally, the viscosity modifier may comprise from about 0.01 to 10 wt %, for example 0.05 wt % to about 5 wt %, e.g. about 0.1 to 3 wt % based on the weight of the total composition. A non-limiting list of suitable viscosity modifiers can be found in the CTFA International Cosmetic Ingredient Dictionary and Handbook, 7^{th} edition, edited by Wenninger and McEwen (The Cosmetic, Toiletry and Fragrance Association, Inc., Washington D.C., 1997).

A wide variety of additional ingredients can be formulated into the hair care compositions according to the present invention. These include: other hair conditioning ingredients such as panthenol, pantethine, pantotheine, panthenyl ethyl ether, and combinations thereof; other solvents such as hexylene glycol; hair-hold polymers such as those described in WO-A-94/08557; viscosity modifiers and suspending agents such as xanthan gum, guar gum, hydroxyethyl cellulose, triethanolamine, methyl cellulose, starch and starch derivatives; viscosity modifiers such as methanolamides of long chain fatty acids such as cocomonoethanol amide; crystalline suspending agents; pearlescent aids such as ethylene glycol distearate; opacifiers such as polystyrene; preservatives such as phenoxyethanol, benzyl alcohol, methyl paraben, propyl paraben, imidazolidinyl urea and the hydantoins; polyvinyl alcohol; ethyl alcohol; pH adjusting agents, such as lactic acid, citric acid, sodium citrate, succinic acid, phosphoric acid, sodium hydroxide, sodium carbonate; salts, in general, such as potassium acetate and sodium chloride; colouring agents; hair oxidising (bleaching) agents, such as hydrogen peroxide, perborate and persulfate salts; hair reducing agents, such as the thioglycolates; perfumes; sequestering agents, such as disodium ethylenediamine tetra-acetate; antioxidants/ultra-violet filtering agents such as octylmethoxycinnamate, benzophenone-3 and DL-alpha tocopherol acetate and polymer plasticizing agents, such as glycerine, diisobutyl adipate, butyl stearate, and propylene glycol. Such optional ingredients generally are used individually at levels from about 0.001 wt % to about 10.0 wt %, preferably from about 0.05 wt % to about 5.0 wt % by weight of the composition.

Mousses, foams and sprays can be formulated with propellants such as propane, butane, pentane, dimethylether, hydrofluorocarbon, CO₂, N₂O, nitrogen or without specifically added propellants (using air as the propellant in a pump spray or pump foamer package).

### Ingredients

The silicon (e.g. when porous) may be loaded with one or more active ingredients. These ingredients include one or more of the following: an anti-dandruff agent or agents, a natural hair root nutrient or nutrients, sunscreen or sunscreens, hair fibre agent or agents, fragrance or fragrances, moisturiser or moisturisers, oil or oils, hair-loss ingredient or ingredients vitamin or vitamins, structural agent or agents, natural active or actives. Typically, the one or more ingredients are present in the range, in relation to the loaded silicon, of 0.01 to 60 wt %, for example 1 to 40 wt % and for example 2 to 10 wt %.

The ingredient to be loaded with the silicon may be dissolved or suspended in a suitable solvent, and silicon particles may be incubated in the resulting solution for a suitable period of time. Both aqueous and non-aqueous slips have been produced from ground silicon powder and the processing and properties of silicon suspensions have been studied and reported by Sacks in Ceram. Eng. Sci. Proc., 6, 1985, pp1109-1123 and Kerkar in J. Am. Chem. Soc. 73, 1990, pp2879-85. The wetting of solvent will result in the ingredient penetrating into the pores of the silicon by capillary action, and, following solvent removal, the ingredient will be present in the pores. Preferred solvents are water, ethanol, and isopropyl alcohol, GRAS solvents and volatile liquids amenable to freeze drying.

In general, if the ingredient to be loaded has a low melting point and a decomposition temperature significantly higher than that melting point, then an efficient way of loading the ingredient is to melt the ingredient.

Higher levels of loading, for example, at least about 15 wt % of the loaded ingredient based on the loaded weight of the silicon may be achieved by performing the impregnation at an elevated temperature. For example, loading may be carried out at a temperature which is at or above the melting point of the ingredient to be loaded. Quantification of gross loading may conveniently be achieved by a number of known analytical methods, including gravimetric, EDX (energy-dispersive analysis by x-rays), Fourier transform infra-red (FTIR), Raman spectroscopy, UV spectrophotometry, titrimetric analysis, HPLC or mass spectrometry. If required, quantification of the uniformity of loading may be achieved by techniques that are capable of spatial resolution such as cross-sectional EDX, Auger depth profiling, micro-Raman and micro-FTIR.

The loading levels can be determined by dividing the volume of the ingredient taken up during loading (equivalent to the mass of the ingredient taken up divided by its density) by the void volume of the porous silicon prior to loading multiplied by one hundred.

### Anti-Dandruff Agents

Suitable examples of anti-dandruff agents include zinc pyrithione, selenium sulphide, tea tree oil, coal tar, sulphur, salicylic acid, 1 hydroxy pyridone. Further examples are the imidazole anti-fungals including miconazole, imidazole, fluconazole, piroctone, clotrimazole, bifonazole, ketaconazole, climbazole, olamine(octopirox), rilopirox, ciclopirox, olamine.

### Sunscreens

Suitable sunscreens include camphor derivatives, benzophenone compounds such as 4,4′-tetrahydroxy-benzophenone which is sold commercially as Uvinul D50, and 2-hydroxy-4-methoxybenzophenone, sold commercially as Eusolex 4360, dibenzoyl methane derivatives such as t-butyl-4-methoxydibenzoylmethane, sold commercially as Parsol 1789, and isopropyldibenzoyl methane, sold commercially as Eusolex 8020. Further suitable types of sunscreen materials are cinnamates, such as 2-ethylhexyl-p-methoxy cinnamate, sold commercially as Parsol MCX, 2-ethoxy ethyl-p-methoxy cinnamate, sold commercially as Giv-Tan F and isoamyl-p-methoxy cinnamate, sold commercially as Neo-Heliopan E1000.

### Natural Hair Root Nutrients

Suitable natural hair root nutrients include amino acids and sugars. Examples of suitable amino acids include arginine, cysteine, glutamine, glutamic acid, isoleucine, leucine, methionine, serine and valine, and/or precursors and derivatives thereof. The amino acids may be added singly, in mixtures, or in the form of peptides, e.g. di- and tripeptides. The amino acids may also be added in the form of a protein hydrolysate, such as a keratin or collagen hydrolysate. Suitable sugars are glucose, dextrose and fructose. These may be added singly or in the form of, e.g. fruit extracts.

### Hair Fibre Benefit Agents

Suitable examples of hair fibre benefit agents include ceramides, for moisturising the fibre and maintaining cuticle integrity. Ceramides are available including by extraction from natural sources, or as synthetic ceramides.

Other suitable materials include fatty acids, for cuticle repair and damage prevention. Particular examples include branched chain fatty acids such as 18-methyleicosanoic acid and other homologues of this series, straight chain fatty acids such as stearic, myristic. and palmitic acids, and unsaturated fatty acids such as oleic acid, linoleic acid, linolenic acid and arachidonic acid.

Split ends may be treated and/or prevented by using a lubricating or plasticizing agent. The surface chemistry of the porous silicon may be adapted to promote hair binding.

### Hair-Loss Ingredients

One or more ingredients suitable for the prevention and/or treatment of hair-loss may be included. Suitable hair loss preventive agents include non-steroidal anti-inflammatories such as piroxicam, ketoprofen, ibuprofen, circulation stimulators such as capsicum or gotu kola, minoxidil or zinc pyridinethione (ZPT), plant extracts such as aloe vera, ginko biloba, olive oil, vitamin E, vitamin B3 and amino acids.

### Head Lice Actives

Suitable actives include insecticides and/or pesticides such as pyrethrins, essential oils, malathion compounds, avermectin compounds.

### Fragrances

Suitable fragrances, or perfuming ingredients, include compounds belonging to varied chemical groups such as aldehydes, ketones, ethers, nitrites, terpenic hydrocarbons, alcohols, esters, acetals, ketals, nitriles. Natural perfuming agents are preferred such as essential oils, resinoids and resins.

With regard to fragrant oils, sustained release may be carried out using mesoporous silicon possessing a pore diameter in the range of about 1-10 nm. The small pore size suppresses the release of the fragrant volatiles.

### Moisturisers

Suitable moisturisers or emollients include glycerine, mineral oil, petrolatum, urea, lactic acid or glycolic acid.

### Oils

Suitable oils include plant oils, essential oils.

### Vitamins

Suitable vitamins include vitamin A, B5, C, E.

### Structural Agents

Suitable structural agents include oils, proteins, polymers that thicken and add body to hair and/or make it feel smoother. Structural agents which add body may be referred to as bulking agents or bulk agent coatings and may be suitable for use with fine hair follicles. These may be colour matched and/or provide a muted glitter appearance with the hair and/or combined with one or more fragrances.

### Natural Actives

Suitable natural actives include herb or plant extracts. Light sensitive plant actives are suitable for use in accordance with the present invention. Entrapment within porous silicon and gradual release provides for improved shelf-life and on-hair photostability.

Mixtures of any of the above active ingredients may also be used.

### (4) Cosmetic Formulations

Cosmetic formulations generally refer to substances or preparations intended for placement in contact with an external part of the human body with a view to providing one or more of the following functions: changing its appearance, altering the odour, cleansing, maintaining/improving the condition, perfuming and protecting.

The silicon may comprise at least one ingredient for delivery to the face or body. Suitable ingredients include one or more of: antioxidants, anti-ageing actives, skin lightening agents, nutrients, moisturisers, antimicrobials, fragrances, oils, vitamins, structural agents, natural actives. The silicon may be loaded with the ingredient which, in the case of porous silicon, may be entrapped in the silicon pores.

The use of silicon containing cosmetic formulations according to the present invention seeks to provide one or more of the following: targeted delivery of ingredients; extended release of ingredients including burst fragrance release, for example, during washing; improved bioavailability of actives, including hydrophobic actives; skin exfoliation; sebum absorption/removal, beneficial degradation products such as orthosilicic acid; retention of significant levels of active ingredients on the body or face over extended periods of time, excellent skin feel and visual appearance.

Suitable antioxidant agents include pycnogenol, plant and fruit extracts, marine extracts, ascorbic acid, glucosides, vitamin E, herbals extracts and synergistic combinations thereof. Suitable anti ageing actives include ceramide, peptides, plant extracts, marine extracts, collagen, calcium amino acids vitamin A, vitamin C and CoQ10. Suitable skin lightening agents include liquorice, arbutin, vitamin C, kojic acid. Suitable moisturisers include panthenol, amino acids, hyaluronic acids, ceramides, sodium PCS, glycerols and plant extracts.

Cosmetic compositions suitable for use in accordance with the present invention may be in the form of creams, pastes, serums, gels, lotions, oils, milks, stick, ointments, powder (including dry powder), solutions, suspensions, dispersions and emulsions.

Suitable cosmetic compositions include: foundation, mascara, nail laquer, nail enamel, deodorant, lipstick, lip balm, lip gloss, colour cosmetics, face cream, eye cream, toner, cleanser, aftersun, moisturiser, shaving cream, after shave, face masks, lip and eye liners, face powder (loose and pressed), eye shadow, bronzer, blush, concealers, face scrub and make up removers. The components comprised in these compositions are well known to the skilled person and these components are suitable for use in the present invention. These components may include a vehicle to act as a carrier or dispersant, emollients, thickeners, opacifiers, perfumes, colour pigments, skin feel components, other sebum absorbing materials, preservatives, mineral fillers and extenders, colour pigments.

In general, cosmetic compositions may contain a vehicle to act as a carrier or dispersant for the silicon so as to facilitate the distribution of the silicon when the composition is applied to the skin. Vehicles other than, or in addition to water can include cosmetic astringents, liquid or solid emollients, emulsifiers, film formers, humectants, skin protectants, solvents, propellants, skin-conditioning agents, solubilising agents, suspending agents, surfactants, ultraviolet light absorbers, waterproofing agents, viscosity increasing agents, waxes, wetting agents. The carrier or dispersant may form about 50 to 90 wt % of the composition. An oil or oily material may be present to provide a water in oil or oil in water emulsion. The compositions may contain at least one active ingredient including skin colourants, drug substances such as anti-inflammatory agents, antiseptics, antifungals, steroids or antibiotics.

Levels of emollients may be 0.5 wt % to 50 wt %, for example 5 to 30 wt %. General classes of emollients include esters, fatty acids, alcohols, polyols, hydrocarbons. Examples of esters include dibutyl adipate, diethyl sebacate, lauryl palmitate. Suitable alcohols and acids include those having from 10 to 20 carbon atoms, for example cetyl, myristyl, palmitic and stearyl alcohols and acids. Examples of polyols include propylene glycol, sorbitol, glycerine. Suitable hydrocarbons include those possessing 12 to 30 carbon atoms, e.g. mineral oil, petroleum jelly, squalene.

A thickener may be present in levels from 0.1 to 20 wt %, for example about 0.5 to 10 wt %. Examples of suitable thickeners include gums e.g. xanthan, carrageenan, gelatin. Alternatively, the thickening function may be provided by any emollient which is present.

Suitable mineral fillers or extenders include chalk, talc, kaolin, mica.

Other minor components may be incorporated into the cosmetic compositions, such as skin feel components. Skin feel components may also include colouring agents, opacifiers and perfumes. These minor components may range from 0.001 wt % to 10 wt %.

Other suitable ingredients may include sebum absorbing materials (other than mesoporous silicon) such as starch, colour pigments, e.g. iron oxides, preservatives such as trisodium EDTA. Other minor components include colouring agents, perfumes, opacifiers which may range from 0.01 to 10 wt %.

Lipstick typically contains pigments, oils, waxes, and emollients and applies colour and texture to the lips. Lip balm is a substance topically applied to the lips of the mouth to relieve chapped or dry lips. Lip gloss is topically applied to the lips of the mouth, but generally has only cosmetic properties. Lip balm may be manufactured from beeswax, petroleum jelly, menthol, camphor, scented oils, and various other ingredients. Other ingredients such as vitamins, alum, salicyclic acid or aspirin may also be present. The primary purpose of lip balm is to provide an occlusive layer on the lip surface to seal moisture in lips and protect them from external exposure. The occlusive materials like waxes and petroleum jelly prevent moisture loss and maintain lip comfort while flavourants, colorants, sunscreens and various medicaments can provide additional, specific benefits. Lip balm usually comes in containers for application with the fingers or in stick form which is applied directly to the lips.

Mascaras can broadly be divided in two groups: water resistant mascaras (often labelled waterproof) and non-water resistant mascaras. Water resistant mascaras have a composition based on a volatile solvent (e.g. isododecane), animal-derived waxes (e.g. beeswax), vegetal based waxes (e.g. carnauba wax, rice bran wax, candelila wax), mineral origin wax (ozokerite, paraffin), pigments (e.g. iron oxide, ultramarine) and film forming polymers. These mascaras do not contain water-sensitive moieties and afford resistance to tears, sweat or rain. Non water-resistant mascaras are based on water, soft surfactants (e.g. triethanolamine stearate), animal-derived waxes (e.g. beeswax), vegetal based waxes (e.g. rice bran wax, candelilla wax), mineral origin waxes (ozokerite, paraffin), pigments (iron oxide, ultramarine), thickening polymers (gum arabic, hydrophobically modified cellulose) and preservatives. These mascaras can run under the effect of tears, but are easily removed with soap and water. Polymers in a water dispersed form (latexes) can bring some level of water resistance to the group of normally non-water resistant mascaras. Waterproof mascaras are similar to oil-based or solvent-based paints. Non water-resistant mascaras behave like water based paints. For intermediate water sensitivity, mascaras contain polymer dispersions.

Face powder is typically applied to the face to set foundation after application. It is absorbent and provides toning to the skin. It can also be reapplied throughout the day to minimize shininess caused by oily skin. There is translucent sheer powder, and there is pigmented powder. Certain types of pigmented facial powders are meant to be worn alone with no base foundation. Powder tones the face and gives an even appearance. Besides toning the face, some SPF based powders can also reduce skin damage from the sun and environmental stress. It comes packaged either as a compact or as loose powder. It can be applied with a sponge, brush, or powder puff. Due to the wide variation among human skin tones, there is a corresponding variety of colours of face powder. There are also several types of powder. A common powder used in beauty products is talc. Commercially available brands may contain natural mineral ingredients. Such products are promoted as being safe and calming for rosacea, as well as improving wrinkles and skin that has been over exposed to sun and has hyper pigmentation. Powdering is a very popular cosmetic technique and is used by many people.

### EXAMPLES

The invention will now be described by way of example only with reference to the following examples.

### Example 1

### Toothpaste Formulations

Mesoporous silicon microparticles are coated with non-porous titania nanoparticles to yield white composite microparticles. The surface chemistries of the silicon and titania particles are chosen to promote co-adhesion. The titania coating is applied by co-dispersing the silicon microparticles in a solution of titania nanoparticles, followed by filtering and drying of the composite powder. The zeta potentials of the silicon and titania surfaces are modified by surface treatments to be preferably high and of opposite polarity. An alternative technique is to exploit electrostatic spraying, wherein the silicon microparticle surface chemistry in aerosol form is such that the surface charge has the opposite polarity to that of the titania nanoparticle aerosol surfaces. The modified particles are not visible when formed into a toothpaste composition.

### Example 2

### Bulking Agent for Food

Metallurgical grade non-porous silicon microparticles are co-ground/milled with a polyol and a natural pigment powder. The white polyol powder is first blended with the pigment. Bulk silicon microparticles with a d₁₀ of 50 μm and d₉₀ of 100 μm are then co-ground to a d₁₀ of 5 μm and d₉₀ of 25 μm to achieve an acceptable “mouthfeel”. During this milling/grinding process freshly cleaved silicon surfaces are created in the presence of the pigment and food component and become coloured at the particle level. The natural pigment powder is chosen from blueberry or mulberry extract to form a purple powder for use in connection with purple chocolate coated sunflower seeds (available from Lyonda Farm, USA); a yellow turmeric pigment provides a grey powder with a hint of green which is used with sage for flavouring sausages and stuffing mixes; a red lycopene pigment produces a brownish red powder for use in tomato paste.

### Example 3

### Blue Hues for Toothpaste

Mesoporous silicon powder of 70 vol % porosity was formed by anodisation, membrane detachment and milling. The resultant microparticles had a d₁₀ of 4 μm, a d₅₀ of 20 μm and a d₉₀ of 44 μm. The powder was then subjected to partial oxidation in air at 800° C. for 3 hours. Blue food-grade dye solution (SuperCook, UK with E133, E122 pigments) was then pipetted onto the powder up to a level before the wet point (where particles clump together due to surface liquid) was reached. The dyes were adsorbed within the mesopores of each microparticle and the resulting colour of the dried powder was a very dark blue. The hue was then adjusted by blending the blue silicon powder with toothpaste-grade titania powder. Weight ratios in the range 1:0.5 to 1:4 (Si:TiO₂) produced blue hues that spanned those typically found in blue striped toothpaste.

### Example 4

### Yellow Powder for Foodstuffs and Beverages

Mesoporous silicon powder of 70 vol % porosity was formed by anodisation, membrane detachment and milling. The resultant microparticles had a d₁₀ of 4 μm, a d₅₀ of 20 μm and a d₉₀ of 44 μm. The powder was then subjected to partial oxidation in air at 800° C. for 3 hours. Curcumin powder was added to ethanol to form a supersaturated solution. The bright yellow solution was slowly pipetted onto 1 g of the porous silicon powder, stirred and maintained at 30° C. to promote ethanol evaporation. Once a uniform yellow colour was achieved, further solution addition was terminated. The dry yellow powder had a weight of 1.02 g indicating 2 wt % pigment content. Its colour was found to match that of various breakfast cereals, and particles were no longer visible when dispersed in orange juice.

### Example 5

### Red Powder for Facial Cosmetics

Mesoporous silicon powder of 70 vol % porosity was formed by anodisation, membrane detachment and milling. The resultant microparticles had a d₁₀ of 4 μm, a d₅₀ of 20 μm and a d₉₀ of 44 μm. The powder was then subjected to partial oxidation in air at 800° C. for 3 hours. Carmine powder was added to ethanol to form a supersaturated solution. 20 ml of this solution was pipetted gradually over 1 g of the mesoporous silicon powder, stirred and maintained at 30° C. to promote ethanol evaporation. A red powder was obtained with less than 1 wt % pigment and was suitable for use in red lipstick.

### Example 6

### Coloration with Fruit and Vegetable Extracts

Fresh blueberries were soaked in methanol within a sealed vessel for 10 hours. The bleached blueberries were then discarded. Oxidised mesoporous silicon powder (65 vol % porosity, hand-milled and oxidised at 800° C.) was then immersed in the dark purple methanol solution on a hot plate for a further 10 hours. Once the methanol had evaporated, further blueberry extract solution was applied and the process repeated to yield a dark burgundy red dry powder.

Commercially available red cabbage extract (Haywards Pickled Red Cabbage, Chivers Hartley Ltd, UK containing red cabbage, water, vinegar, salt and spice extracts) was used to “stain” oxidized mesoporous silicon by repeated immersion and drying. The resultant silicon powder was dark purple in colour.

Fresh kale was shredded in a kitchen blender with propanol and the alcohol suspension then ground by hand with a pestle and mortar. The mixture was then filtered to remove leaf sediment. The green solution containing natural chlorophyll pigment was then pipetted onto both white porous silica powder and powder that had been partially reduced to porous silicon using magnesium vapour (a very pale brown powder). The green hue, after solvent removal by evaporation, can be tuned by varying the degree of silica reduction.

### Example 7

### Red Powder for Foodstuffs

A red natural anthocyanin based pigment was loaded into mesoporous silica powder (containing no elemental silicon and for the purposes of comparison) and mesoporous silicon powder that had been thermally oxidised for 3 hours at 800° C. The two resulting red powders were then both used to colour the surface of a planar confectionary product. The foodstuff was then subjected to 7 mW/cm² longwave 325 nm UV light for 20 hours at 40° C. The foodstuff items coloured with the mesoporous silicon carrier coating underwent significantly less fading than those coloured with the pigmented mesoporous silica carrier. Further pieces of the same foodstuff were subjected to 10 minutes of 1.8 mW/cm², 254 nm shortwave UV light at 40° C. The oxidised mesoporous silicon coating showed less fading and improved photoprotection of the pigment compared with the silica coating.

1. A method for masking the appearance of particulate elemental silicon in a composition, comprising modifying the colour of the particulate elemental silicon so that the silicon is not distinguishable to the human eye when blended with the other components of the composition.

2. A method according to claim 1, wherein the silicon is modified prior to blending with the other components.

3. A method according to claim 1, wherein the silicon comprises one or more of amorphous silicon, single crystal silicon and polycrystalline silicon.

4. A method according to claim 1, wherein the silicon is porous silicon.

5. A method according to claim 4, wherein the porous silicon is selected from one or more of microporous silicon, mesoporous silicon or macroporous silicon.

6. A method according to claim 4, wherein the porous silicon comprises or consists essentially of surface modified silicon.

7. A method according to claim 6, wherein the surface modified porous silicon comprises or consists essentially of or consists of one or more of derivatised porous silicon, partially oxidised porous silicon, porous silicon modified with silicon hydride surfaces.

8. A method according to claim 7, wherein the surface modified porous silicon comprises or consists essentially of or consists of partially oxidised porous silicon.

9. A method according to claim 8, wherein the partially oxidised porous silicon possesses an oxide content corresponding to between about one monolayer of oxygen and a total oxide thickness of less than or equal to about 4.5 nm covering the entire skeleton.

10. A method according to claim 1, wherein the colour of the silicon is modified by a masking material in contact with the silicon.

11. A method according to claim 10 and wherein the silicon is selected from porous silicon and wherein the masking material fills some, or all, or substantially all of the pores.

12. A method according to claim 11 wherein the masking material coats the porous silicon and forms a capping layer.

13. A method according to claim 10, wherein the masking material is suitable for delivery to the human or animal body.

14. A method according to claim 10, wherein the masking material comprises a hydrophobic nutrient.

15. A method according to claim 10, wherein the masking material comprises an ingredient that is present in the composition.

16. A method according to claim 10, wherein the masking material comprises a natural or nature-identical ingredient.

17. A method according to claim 1, wherein the silicon is non-porous silicon which is modified to a colour other than dark grey or black.

18. A method according to claim 1, wherein the silicon is porous silicon which is modified to a colour other than dark brown or light brown.

19. A method according to claim 1, wherein the silicon is loaded with at least one ingredient for delivery to the human or animal body and wherein the ingredient and masking material are different.

20. A method according to claim 1, wherein the colour of the silicon is modified by controlling one or more of the porosity, surface roughness, the arrangement of individual particles, the arrangement and size of individual particles, the silicon oxide content.

21. A method according to claim 15, wherein the colour of the silicon is modified by arranging nanoparticles of silicon into microparticle clusters wherein the cluster comprises at least 100,000 nanoparticles of silicon.

22. A method according to claim 1, wherein the composition is chosen from a food composition, an oral hygiene composition, a hair care composition, a cosmetic composition.

23. Use of partially oxidised porous silicon as a photoprotector for a masking agent in a composition.

24. Use according to claim 23 wherein the porous silicon is mesoporous silicon.

25. Use according to claim 23, wherein the partially oxidised porous silicon possesses an oxide content corresponding to between about one monolayer of oxygen and a total oxide thickness of less than or equal to about 4.5 nm covering the entire skeleton.

26. Use according to claim 23, wherein the masking agent is loaded in the pores of the partially oxidised porous silicon.

27. Use according to claim 26, wherein the masking agent is a white or coloured pigment.
